(19) United States
(12) Patent Application Publication
(10) Pub. No.: US 2025/0208139 A1
(43) Pub. Date: Jun. 26, 2025

(54) METHOD FOR RAPID IMMUNOHISTOCHEMICAL DETECTION OF AN ANTIGEN FROM A BIOLOGICAL SAMPLE

(71) Applicants: Marc Key, Ojai, CA (US); Diagnostic BioSystems, Pleasanton, CA (US)

(72) Inventors: Marc Key, Ojai, CA (US); Bipin Gupta, Pleasanton, CA (US)

(73) Assignees: Marc Key, Ojai, CA (US); Diagnostic BioSystems, Pleasanton, CA (US)

(21) Appl. No.: 19/081,829

(22) Filed: Mar. 17, 2025

Related U.S. Application Data
(63) Continuation-in-part of application No. 17/516,829, filed on Nov. 2, 2021.
(60) Provisional application No. 63/139,921, filed on Jan. 21, 2021.

(51) Int. Cl. G01N 33/574; G01N 33/58

(52) U.S. Cl. CPC G01N 33/57484; G01N 33/581; G01N 2474/20

(57) ABSTRACT

A method to produce a rapid immunohistochemical detection of an antigen from a biological sample is provided. Preferably, the method may be used for rapid immunohistochemical staining of a biological sample that allows completion of the staining process in less than ten minutes, such as during a cancer removal procedure. Preferably, the method may include the steps of: depositing a section of the biological sample on a slide; permeabilizing the section of the biological sample; incubating the section of the biological sample with a secondary antibody having a detectable label; removing unbound secondary antibody from the section of the biological sample; mounting the section of the biological sample; and detecting secondary antibody bound to the section of the biological sample.

### CROSS REFERENCE TO RELATED APPLICATIONS

This application is a continuation-in-part of U.S. Non-Provisional application Ser. No. 17/516,829, filed on Nov. 2, 2021, entitled “METHOD FOR RAPID IMMUNOHISTOCHEMICAL DETECTION OF AN ANTIGEN FROM A BIOLOGICAL SAMPLE”, which claims priority to and the benefit of the filing date of U.S. Provisional Application No. 63/139,921, filed on Jan. 21, 2021, entitled “Rapid Stain for Tissue Proteins”, which is hereby incorporated by reference in its entirety.

### FIELD OF THE INVENTION

The present invention relates generally to a method of staining a biological sample and more specifically it relates to a method of staining and examining a biological sample to identify the presence of tumor cells in the biological sample.

### BACKGROUND

Biological samples of cells and/or tissues are obtained for purposes of analyzing the biological constituents that comprise the sample. Sample analysis may be performed by methods of immunohistochemistry for analyzing protein components, or in situ hybridization for analyzing nucleic acid components. Prior to the analysis the sample must be prepared by appropriate methods. For example, a tissue biopsy is removed from a patient and processed by a standard histological method for embedding the biopsy into a solid block of paraffin. The tissue is embedded into a solid block to provide a firm surrounding matrix to facilitate sectioning. Briefly the tissue biopsy is surgically removed and placed into a fixative, such as formalin. Other fixatives may also be used but formalin in the most common. The formalin is typically used at about 10% in a buffered solution. Fixation time may be any time between 2-48 hours. A fixation time of about 8-24 hour is typical. The fixed tissue is then dehydrated through a series of alcohols and then the alcohol is removed and replaced with a paraffin solvent such as xylene. The alcohol is removed from the tissue by placing the tissue into successive baths containing increasing concentrations of xylene until the tissue is in 100% xylene. The xylene is then removed and replaced with paraffin by placing the tissue into successive baths with increasing concentrations of paraffin. The baths must be kept above the melting point of paraffin (about 60 C) to keep the solutions from solidifying. After the tissues are in 100% paraffin, they are formed into blocks and allowed to cool. As the cooling proceeds the blocks containing the tissues solidify. Once solidified the blocks containing the tissues are placed into a microtome that cuts thin sections of the tissues. Each thin section is about 4 u in thickness. The thin sections are applied to a microscope slide in preparation for immunohistochemical staining. The process so described is the most common method for preparing a tissue for microscopic examination. However, the process is time consuming generally requiring several hours to complete. Consequently, an alternative method may be employed in situations where short times are critical. In contrast to utilizing paraffin-embedded tissues, a shortened method for preparing a biopsy relies on the use of frozen tissues. The frozen tissue method has the following steps:
1. Fixation: Tissue is fixed to stop all metabolic activity and to preserve the molecular structure of the tissue. This may include chemical fixation, such as formalin, or fixation by freezing. The frozen tissue can be flash frozen in liquid nitrogen or a low temperature bath of isopentane and dry ice. The solid frozen tissue can then be embedded into a block and attached onto a holder frequently referred to as a chuck. The holder is typically a metal disc onto which the tissue is placed. An embedding medium may be used in order to attach the frozen tissue to the chuck. The tissue is now ready for sectioning on a cryostat. The cryostat is a device that can cut thin sections from the frozen tissue while maintaining a sub-zero temperature, such as −20 C. The tissue is typically sectioned at about 4-8 u to produce very thin tissue sections. The tissue sections are then placed onto a cold microscope slide where they become adherent. Typically, the microscope slides will have a charged surface that aids in tissue adherence. The microscope slides with adherent tissue are ready for pre-treatment in preparation for immunohistochemical staining.
2. Immunohistochemical Staining with Primary Antibody: The tissues are ready for immunohistochemical staining. For all immunohistochemical staining steps an appropriate buffer must be used. The typical buffer would be similar to physiological conditions in terms of molarity and pH. These conditions favor the binding of antibodies to antigens and enzyme-substrate reactions.

The first step in the staining process is to apply the primary antibody in a suitable buffer solution. The antibody is selected to bind to the antigen of interest, where the antigen of interest may be a protein that helps identify the particular tissue or tumor under investigation. If the primary antibody binds, then the antigen of interest is present, whereas if the primary antibody does not bind, then the antigen of interest is absent. A positive binding event will allow identification of the tumor cells. The primary antibody is applied to the tissue and incubated for a sufficient length of time to allow binding to occur.

The primary antibody is selected to bind to the antigen of interest. The primary antibody is typically generated by immunizing an animal with the antigen of interest. Commonly primary antibodies are produced in mice or in rabbits, although other animals can also be used. Typically, the antibodies are monoclonal antibodies, where the antibody is generated from a single clone of antibody-producing cells, or the antibodies may be polyclonal where the antibodies are produced from multiple different antibody-producing cells. A polyclonal antibody may be harvested from the serum of the immunized animal and in this state may be referred to as antiserum.
3. Immunohistochemical Staining with Tagged Secondary: The second step in this rapid immunohistochemistry process is to determine whether a binding event of primary antibody to antigen has occurred. In order to make this determination a secondary antibody is next applied. The secondary antibody has several unique features that allow it to be used in this respect. First the secondary antibody must be specific for the first primary antibody. For example, if the primary antibody has been produced in a mouse, then the secondary antibody must be anti-mouse immunoglobulin (Ig) specific. If the primary antibody has been produced in a rabbit then the secondary antibody must by anti-rabbit Ig specific. The second feature of the secondary antibody is that it must have a detectable tag such as an enzyme. Commonly used enzymes in immunohistochemistry include peroxidase and alkaline phosphatase.
4. Immunohistochemical Staining for Detection: After binding of the secondary antibody to the primary antibody the complex now contains a detectable label such as an enzyme. Common enzymes include alkaline phosphatase and peroxidase, most commonly peroxidase from horseradish peroxidase. There are numerous substrate/chromogen combinations that can be used with these enzymes that produce an insoluble colored reaction product that is deposited onto the microscope slide at the location where the primary antibody has bound. The presence of a colored reaction product is indicative of a positive test, meaning that the primary antibody has bound to its antigen. A common chromogen for use with alkaline phosphatase includes Fast Red which produces a red reaction product, and a common chromogen for use with peroxidase includes diaminobenzidine (DAB) which produces a brown reaction product at the site of an antigen-antibody reaction.

Once the secondary antibody has bound to the primary antibody, a detectable enzyme is now present. The next incubation step is to add a suitable substrate/chromogen mixture. The resultant enzyme-substrate reaction results in a chromogen being converted from a colorless form to a colored form. Furthermore, the chromogen is insoluble and deposits as a precipitate at the site of the enzyme. This colored reaction product is visible microscopically and is indicative of a positive reaction. After completion of the immunohistochemical stain, the tissue may be further stained with a counterstain. The counterstain will stain all the tissue elements whether or not they also have an immunohistochemical stain. The counterstain is useful for studying the overall structure and morphology of the tissue as a whole. Thus, the immunohistochemical stain provides a molecular stain and the counterstain provides a morphological stain. The counterstain would be chosen such that it would have a different color from the immunohistochemical stain so that it does not obscure the stain.
5. Preparing the Slides for Microscopic Examination: Having completed the immunohistochemical stain and the counterstain, the final steps include preparing the slides for microscopic examination. Preferred mounting procedures utilize resin-based mounting medium often referred to as permanent mounting medium. These mounting mediums contain resins and plastic dissolved in an organic solvent such as toluene or xylene. Because these mounting mediums are insoluble with water, all the water in the tissues must be removed prior to mounting. In a typical mounting procedure, the stained tissues are dehydrated in a series of alcohols beginning with a mixture of water and alcohol and gradually moving through a series of baths with decreasing water and increasing alcohol until the slides are in a bath of 100% alcohol. Next the alcohol is gradually removed and replaced with a solvent such as xylene or toluene by moving the slides through a series of baths beginning first with a bath of alcohol and solvent and then moving through a series of baths with decreasing alcohol and increasing solvent until the slides are in 100% solvent. Finally at this stage the slides are ready for mounting. The slides are removed from the last solvent bath, and a drop of mounting medium is applied. Next a glass coverslip is overlaid the mounting medium. The mounting medium dries and glues the coverslip over the tissue. The tissues are now ready for microscopic examination.

Even though the frozen tissue method of preparing a tissue is much faster than the paraffin-embedded method, neither method is fast enough to meet the requirements for fast turn around time from sample collection to results. Therefore, a need exists for novel methods to be used for providing rapid immunohistochemical detection of an antigen from a biological sample.

### Mohs Surgery

Mohs surgery is a type of surgery for the diagnosis and treatment of a presumptive skin lesion suspected of being cancer.

Mohs surgery is a microscopically controlled procedure that removes skin cancer layer by layer. It is a highly effective treatment for many types of skin cancer such as carcinomas and melanomas. The surgeon removes thin layers of skin and examines each layer under a microscope for cancer cells. Each skin layer is stained with two dyes called hematoxylin and eosin (H&E) to reveal the presence of cancer cells. If cancer cells are identified in the stained skin sample, then the process is continued until there are no signs of cancer in the stained tissue. The Mohs surgery and staining method is designed to be a rapid procedure that can be concluded while the patient remains on the operating table. If the surgery is successful the patient can go home following the procedure and no further treatment is necessary. However, if the staining method fails to identify a few remaining tumor cells in the skin sample, then the patient will be required to return for further treatment or the tumor will eventually recur. To prevent such events an improved method for staining and identifying residual tumor cells in Mohs skin samples is needed.

Mohs surgery is used for treatment of skin cancer and is performed by excision of the malignant tissues. The skin tissue removed during surgery is stained to identify the type of tumor, such as squamous cell carcinoma, basal cell carcinoma or malignant melanoma. Additionally, the tissue is analyzed to determine the amount of cancer and the extent of invasion of the cancer cells. Both the diagnosis and the evaluation of the extent of invasion are based on the morphological appearance of the cancer cells in a standard H&E stain.

The Mohs surgery is intended to be a rapid procedure that can be completed in a single operation without requiring the patient to return for subsequent treatments. This is accomplished by removing and examining layers of skin and underlying tissues until the tissue shows no further evidence of tumor cell invasion. If the tissue samples are negative for tumor cells, it is assumed that all cancerous tissues have been successfully removed. The microscopic assessment of the tissues is based on morphologic criteria which are used to identify both the type of the cancer and the extent of invasion. However, morphological assessment alone can lead to inaccuracies, particularly when minimal tumor is present and residual tumor is obscured by an abundance of normal tissue elements. Therefore, a more accurate method of identifying the presence of rare tumor cells is needed.

### Immunohistochemistry As A Supplemental Staining Method

Immunohistochemistry (IHC) is a staining method that has the ability to accurately stain and identify rare tumor cells in a tissue sample even down to the single cell level.

Immunohistochemistry is a method that has been shown to improve the accuracy of evaluation of Mohs surgical samples. When compared to standard Mohs staining with H&E, the IHC method was more sensitive in identifying rare tumor cells. However, IHC is rarely performed as part of the initial Mohs procedure. This is because IHC is a relatively long staining method that cannot be used at the time of initial surgery. When IHC is utilized as an aid in evaluating skin samples, it is not used concurrently with the Mohs surgery but is used as a follow-up staining method where the results of such follow-up testing may take several days to complete. Consequently if rare tumor cells are identified in the IHC stain, the patient will likely have to be recalled for further surgery. If a more rapid method of IHC staining were available, then the benefits of IHC staining could be run as part of the Mohs surgery staining. This would result in a more accurate initial evaluation of the skin layers for residual cancer and less likelihood of needing to call patients back for subsequent surgery.

IHC staining methods identify cells by both morphological staining characteristics as well as molecular profile of the cells. Cancer cells possess certain protein molecules that can serve as markers for their definitive identification. These molecular markers serve as targets for the IHC methods. By utilizing specific antibodies, these target molecules can be made to stain various colors whereas the normal tissue elements remain uncolored. For example, melanoma cancer cells can be stained by the IHC method to stain brown whereas the normal cells will stain another color, typically, blue, providing a striking color contrast between the tumor cells and the normal cells. During microscopic evaluation the brown tumor cells can be easily distinguished from the normal blue cells even down to a single tumor cell among several hundred normal cells.

The most common way for IHC staining to be used in conjunction with Mohs surgery, is to send a sample of the tissue remaining after Mohs surgery to a central histology laboratory for processing and staining. These methods take several hours or days to complete. Standard methods of IHC require fixation, embedding, sectioning, staining, and evaluation. Another much faster method of IHC staining could use frozen tissues, thus eliminating the steps of fixation and embedding. However, the remaining steps are still laborious and time-consuming and cannot be used simultaneously with Mohs surgery. What is needed is a rapid stain for IHC that can be used concurrently with Mohs surgery and standard Mohs procedures.

### BRIEF SUMMARY OF THE INVENTION

A method to produce a rapid immunohistochemical detection of an antigen from a biological sample is provided. Preferably, the method may be used for rapid immunohistochemical staining of a biological sample that allows completion of the staining process in less than ten minutes. Such rapid staining is essential in certain diagnostic settings. For example, in diagnosis of melanoma the patient may be required to remain in surgery until the staining and diagnosis is completed.

In some embodiments, the method may include the steps of: depositing a section of the biological sample on a slide; permeabilizing the section of the biological sample; incubating the section of the biological sample with a secondary antibody having a detectable label; removing unbound secondary antibody from the section of the biological sample; mounting the section of the biological sample; and detecting secondary antibody bound to the section of the biological sample.

In further embodiments, the method may be used during a cancer removal procedure and the method may include the steps of: depositing a section of the biological sample on a slide, in which the section of the biological sample is produced by freezing the biological sample and then sectioning the biological sample; permeabilizing the section of the biological sample; incubating the section of the biological sample with a secondary antibody having a detectable label, in which the secondary antibody is specific to a Fc portion of a primary antibody also applied to the section of the biological sample; removing unbound secondary antibody from the section of the biological sample; mounting the section of the biological sample, in which the step of mounting the section of the biological sample includes applying a mixture of an alcohol and an alcohol and water soluble polymer to the section of the biological sample; and detecting secondary antibody bound to the section of the biological sample.

It is an object of the present invention to produce a method for rapid immunohistochemical stain of antigens within a biological sample.

Another object is to provide a rapid stain for tissue proteins that uses a method to permeabilize the tissue.

Another object is to provide a rapid stain for tissue proteins that utilizes an Fc-specific enzyme conjugate to detect and bind to the primary antibody.

Another object is to provide a rapid stain for tissue proteins that utilizes a mixture of a primary antibody and a secondary antibody where the secondary antibody is an Fc-specific enzyme conjugate.

Another object is to provide a rapid stain for tissue proteins that utilizes method for rapid dehydration of the immunohistochemically stained biological sample.

Another object is to provide a rapid stain for tissue proteins that utilizes a method and reagent to permanently seal the immunohistochemically stained biological sample.

An object is to provide a method of staining a biological sample for providing information relating to the presence of tumor cells in the biological sample.

Another object is to provide a method of staining a biological sample that contains a malignancy of the skin and is obtained by means of Mohs surgery.

Another object is to provide a method of staining a biological sample that is stained with a first stain and then evaluated microscopically where a determination is made as to which of two second stains shall be applied.

Another object is to provide a method of staining a biological sample where the second stain is either comprised of eosin (the Mohs stain) or the second stain is an immunohistochemical stain.

Another object is to provide a method of staining a biological sample that utilizes a second stain that is a rapid immunohistochemistry stain.

### BRIEF DESCRIPTION OF THE DRAWINGS

Some embodiments of the present invention are illustrated as an example and are not limited by the figures of the accompanying drawings, in which like references may indicate similar elements and in which:

FIG. 1 depicts a block diagram of an example of a method to produce a rapid immunohistochemical detection of an antigen from a biological sample according to various embodiments described herein.

FIG. 2 illustrates a block diagram of an example of a method to rapidly permeabilize a biological sample according to various embodiments described herein.

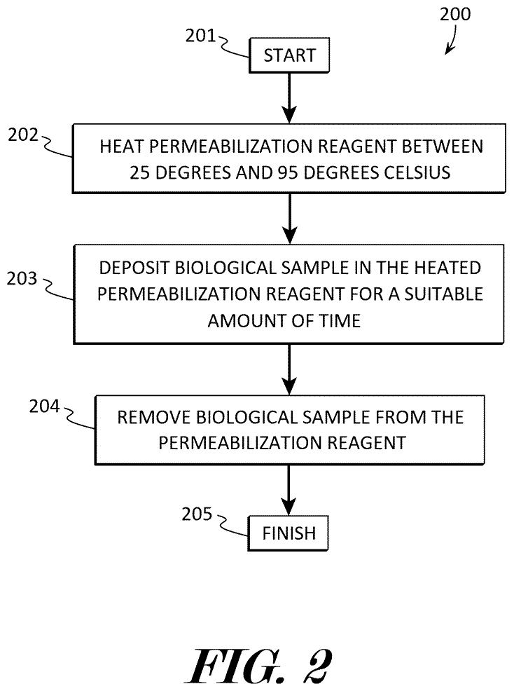

FIG. 3 shows a block diagram of an example of a method to rapidly bind an antigen of a biological sample with a labeled secondary antibody according to various embodiments described herein.

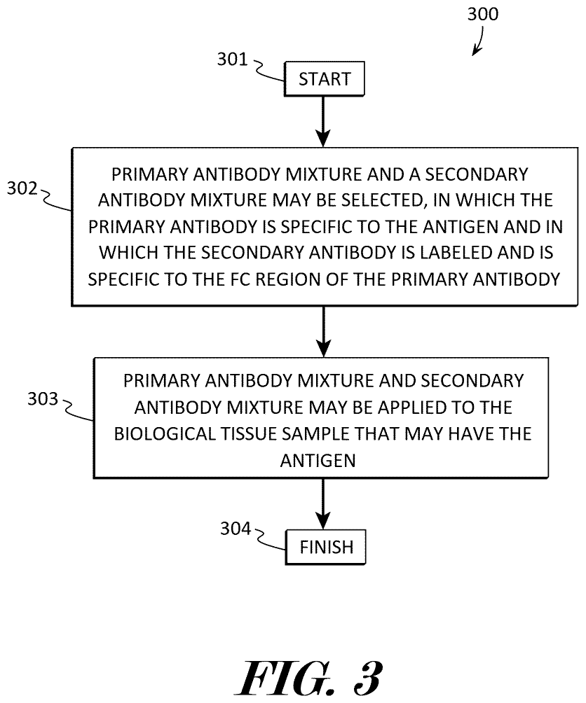

FIG. 4 depicts a block diagram of an example of a method of rapidly dehydrating a biological sample according to various embodiments described herein.

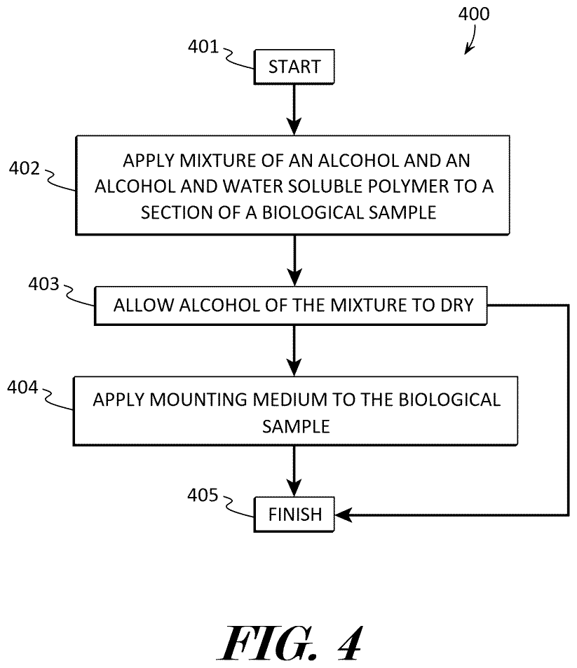

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

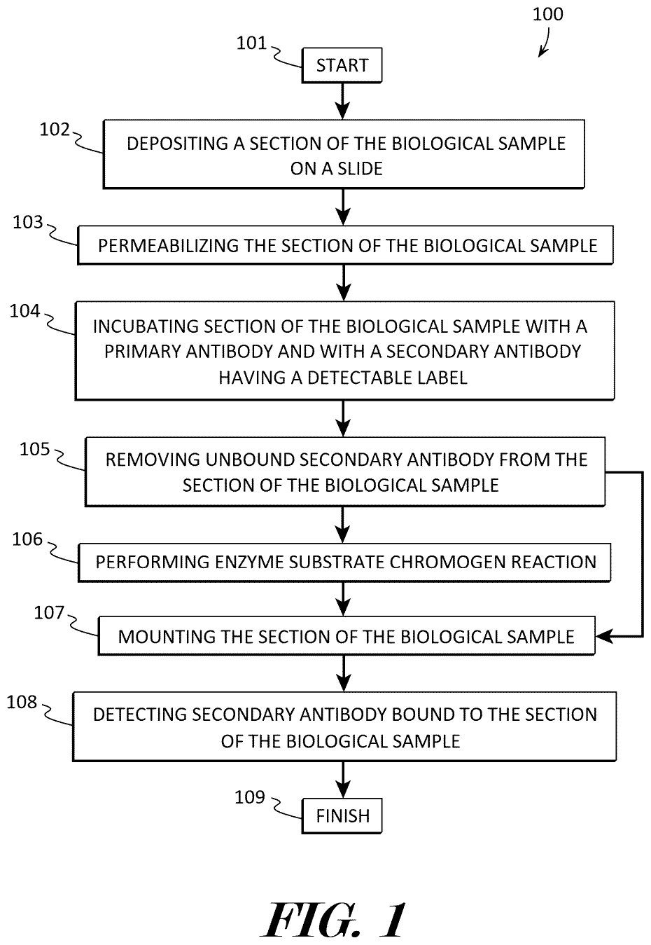

For purposes of description herein, the terms “upper,” “lower,” “left,” “right,” “rear,” “front,” “side,” “vertical,” “horizontal,” and derivatives thereof shall relate to the invention as oriented in FIG. 1. However, one will understand that the invention may assume various alternative orientations and step sequences, except where expressly specified to the contrary. Therefore, the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Although the terms “first,” “second,” etc. are used herein to describe various elements, these elements should not be limited by these terms. These terms are only used to distinguish one element from another element. For example, the first element may be designated as the second element, and the second element may be likewise designated as the first element without departing from the scope of the invention.

As used in this application, the term “about” or “approximately” refers to a range of values within plus or minus 10% of the specified number. Additionally, as used in this application, the term “substantially” means that the actual value is within about 10% of the actual desired value, particularly within about 5% of the actual desired value and especially within about 1% of the actual desired value of any variable, element or limit set forth herein.

A new method for rapid immunohistochemical detection of an antigen from a biological sample is discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

The present invention will now be described by example and through referencing the appended figures representing preferred and alternative embodiments. FIG. 1 illustrates an example of a method for rapid immunohistochemical detection of an antigen from a biological sample (“the method”) 100 according to various embodiments. In some embodiments, the method 100 may be used for the rapid immunohistochemical staining of a biological sample that allows completion of the staining or detection process in less than ten minutes. Such rapid antigen detection is essential in certain diagnostic settings. For example, in diagnosis and removal of melanoma the patient may be required to remain in surgery until the detection and diagnosis is completed. In preferred embodiments, the method 100 may be a method for rapid immunohistochemical detection of an antigen from a biological sample during a cancer removal procedure, such as during Moh's micrographic surgery for skin cancer removal.

In some embodiments, the method 100 may start 101 and a section of the biological sample may be deposited on a slide is step 102. Preferably the biological sample may have been or may be fixed to stop all metabolic activity and to preserve the molecular structure of the tissue of the biological sample.

In some embodiments of the method 100, the section of the biological sample may be produced by embedding the biological sample in paraffin or other suitable embedding medium that may provide a firm surrounding matrix to facilitate sectioning and then the embedded biological sample may be sectioned. During conventional cancer removal procedures, such as Moh's micrographic surgery, the tissue is prepared and embedded into a paraffin block by standard histological methods. Briefly the tissue biopsy is surgically removed and placed into a fixative, such as formalin. Other fixatives may also be used but formalin in the most common. The formalin is typically used at about 10% in a buffered solution. Fixation time may be any time between 2-48 hours. A fixation time of about 8-24 hour is typical. The fixed tissue is then dehydrated through a series of alcohols and then the alcohol is removed and replaced with a paraffin solvent such as xylene. The alcohol is removed from the tissue by placing the tissue into successive baths containing increasing concentrations of xylene until the tissue is in 100% xylene. The xylene is then removed and replaced with paraffin by placing the tissue into successive baths with increasing concentrations of paraffin. The baths must be kept above the melting point of paraffin (about 60 degrees Celsius) to keep the solutions from solidifying. After the tissues are in 100% paraffin, they are formed into blocks and allowed to cool. As the cooling proceeds the blocks containing the tissues solidify. Once solidified the blocks containing the tissues are placed into a microtome that cuts thin sections of the tissues. Each thin section is about 4 micrometers in thickness. The thin sections are applied to a microscope slide in preparation for immunohistochemical staining.

In preferred embodiments of the method 100, the section of the biological sample may be produced by freezing the biological sample and then sectioning the biological sample. For rapid immunohistochemistry it is preferable to freeze the tissue rather than embed the tissue in paraffin because the time required to process a frozen tissue is significantly shorter than the time required to embed a tissue in paraffin. The frozen tissue can be flash frozen in liquid nitrogen or a low temperature bath of isopentane and dry ice. The solid frozen tissue can then be embedded into a block and attached onto a holder frequently referred to as a chuck. The holder is typically a metal disc onto which the tissue is placed. An embedding medium may be used in order to attach the frozen tissue to the chuck. The tissue is now ready for sectioning on a cryostat. The cryostat is a device that can cut thin sections from the frozen tissue while maintaining a sub-zero temperature, such as −20 C. The tissue is typically sectioned at about 4-8 micrometers to produce very thin tissue sections. The tissue sections are then place onto a cold microscope slide where they become adherent. Typically, the microscope slides will have a charged surface that aids in tissue adherence. The microscope slides with adherent tissue are ready for pre-treatment in preparation for immunohistochemical staining.

In step 103 the section of the biological sample may be permeabilized. In preferred embodiments, the microscope slide with the attached tissue is brought to room temperature, at which time the tissue melts and attaches to the microscope slide. Based on the molecular structure of the tissue and the specific antigens under investigation, the antigens may be hidden or otherwise inaccessible to the primary antibody. Therefore, it has been found to be useful to first permeabilize the tissue prior to staining. Although the exact mechanism by which the permeabilization reagent exposes hidden antigens is unknown it is thought to act by changing the molecular structure of the tissue and antigens. The detergent contained in the permeabilization reagent may destroy or dissolve lipid layers and other hydrophobic molecules thereby allowing the hydrophilic antibodies to more easily penetrate the tissues. Furthermore, chelating agents may be useful in helping to stabilize proteins.

In some embodiments, the permeabilization reagent preferably is approximately a 0.05 M Tris (tris (hydroxymethyl) aminomethane) buffer containing about 5-10 mg/ml of ethylenediaminetetraacetic acid (EDTA) and about 1% detergent (plus or minus 0.5%) such as Tween 20.

In some embodiments, permeabilization procedures may be performed by submerging slides with attached tissues into the permeabilization reagent that is at room temperature, such as between 17 and 20 degrees Celsius, and incubated for a sufficient length of time that the tissues are permeabilized. The length of time necessary for permeabilization varies depending on the temperature of the incubation mixture. In some embodiments, after about 1 minute (plus or minus 10 seconds) in the permeabilization reagent the slides may be removed and rinsed with a 0.05 M Tris buffer.

In preferred embodiments of step 103, the section of the biological sample may be permeabilized via a method to rapidly permeabilize a biological sample (“the method”) 200. In some embodiments, the method 200 may start 201 and a permeabilization reagent may be heated to between 25 degrees and 95 degrees Celsius in step 202. In preferred embodiments, the permeabilization reagent may be heated to approximately 90 degrees Celsius. Next in step 203, the biological sample may be deposited in the heated permeabilization reagent for a suitable amount of time, such as between 10 seconds and 10 minutes. In preferred embodiments, the permeabilization reagent may be heated to approximately 90 degrees Celsius, and the biological sample may be deposited in the heated permeabilization reagent for between approximately 30 seconds to 90 seconds, and more preferably for approximately one minute. Furthermore, because of the short incubation time, the entire permeabilization step can be conducted at room temperature with only slight cooling of the permeabilization reagent. Next in step 204, the biological sample may be removed from the permeabilization reagent. After step 204, the method 200 may finish 205.

Permeabilization in step 103 may also be conducted at different temperatures, but the incubation times may have to be increased to achieve sufficient permeabilization. For example, sufficient permeabilization will occur even under conventional room temperature incubation conditions if the incubation time is extended to 60 minutes.

For rapid immunohistochemistry the permeabilization reagent may be pre-heated in step 202, such as in a microwave for about one minute, in order to achieve a temperature of about 90 degrees Celsius. Pre-heating can take place while the microscope slides are being sectioned, thus pre-heating does not add any time to the overall rapid immunohistochemistry staining process. Once the slides are placed into the heated permeabilization reagent in step 203, they are incubated for about one minute in a room temperature environment and then removed from the permeabilization reagent in step 204. After removal from the permeabilization reagent, the slides having the biological samples may be transferred to a buffer bath that is suitable for immunohistochemistry such as a Tris or Phosphate buffer at about pH 7.2.

Following permeabilization the tissues are ready for immunohistochemical staining. For all immunohistochemical staining steps an appropriate buffer must be used. The typical buffer would be similar to physiological conditions in terms of molarity and pH. These conditions favor the binding of antibodies to antigens and enzyme-substrate reactions.

In step 104 the biological sample may be incubated with a primary antibody and with a secondary antibody having a detectable label to determine whether a binding event of a primary antibody to an antigen of interest has occurred. A secondary antibody used in step 104 preferably may have several unique features that allow it to be used in this respect. First, the secondary antibody must be specific for a primary antibody applied to the biological sample that is specific to the antigen of interest that may be in the biological sample. For example, if the primary antibody has been produced in a mouse, then the secondary antibody must be anti-mouse immunoglobulin (Ig) specific. If the primary antibody has been produced in a rabbit, then the secondary antibody must by anti-rabbit Ig specific. The second feature of the secondary antibody is that it must have a detectable label or tag, such as an enzyme molecule, a polymer having two or more enzyme molecules bound to it, a fluorescent tag, etc. Commonly used enzymes in immunohistochemistry include peroxidase and alkaline phosphatase. Furthermore, enzymes may be directly attached to the secondary antibody or attached via a polymer backbone. The advantage of using a polymer backbone is that multiple same and/or different enzyme molecules and multiple same and/or different secondary antibodies can be contained within a polymerized molecular structure, thus providing increased sensitivity to the detection method. Antibody polymers can be produced by conjugating multiple antibody molecules to a polymer backbone. Additionally, multiple enzyme molecules can be conjugated to the antibody-polymer structure. In preferred embodiments, the method 100 may use polymers of antibody and two or more enzyme molecules in the performance of step 104 because they provide greater sensitivity compared to antibodies having a single enzyme molecule.

In preferred embodiments, step 104 may utilize a secondary antibody that is Fc-specific to a primary antibody also applied to the biological sample. Fc-specific antibodies have unique specificity in that they bind to only a certain region of a primary antibody and this region is termed the Fc portion. The Fc portion of an antibody is not involved in the binding of an antibody to its antigen. The antigen combining regions are termed Fab. Therefore, at one end of the antibody molecule are the Fab regions and on the other end of the antibody molecule are the Fc regions. A secondary antibody that is not Fc-specific will bind to all regions of the primary antibody including the Fab and Fc regions.

In conventional immunohistochemistry the primary antibody is incubated with the tissue for a sufficient length of time in order for the antibody to bind to its antigen. After a sufficient length of time has passed the tissue is rinsed such that all unbound primary antibodies are removed. Any bound primary antibodies will remain attached to the tissue. Next a secondary antibody is applied and incubated for a sufficient length of time that it will bind to its primary antibody, if present. However, in the present invention the primary antibody and the secondary antibody can be incubated together at the same time and/or the secondary antibody can be added to the slide at any point after the primary antibody without the requirement to first rinse off unbound primary antibody. The incubation of the primary antibody and secondary antibody can be performed in a variety of ways. The primary antibody and secondary antibody can be premixed, and then applied to the tissue as a single reagent. The primary antibody can be applied to the tissue and allowed to react for a short period of time, and then without rinsing the secondary antibody can be directly added to the incubation mixture. And finally, the primary antibody can be incubated for a short period of time, the excess reagent drained from the slide without rinsing, and then the secondary antibody added to the remaining incubation mixture.

In the case where the primary antibody and Fc-specific secondary antibody are premixed before adding to the tissue, the primary antibody would typically be used at a concentration of about 2-5 ug/ml, and the Fc-specific secondary antibody would be used at a concentration of about 5-15 ug/ml. As a general rule the ratio of the primary antibody to Fc-specific secondary antibody would be about 1:3 weight by weight (w/w) [e.g., 1:2.5 to 1:3.5 (w/w)]. The complex of primary and secondary antibody is prepared in a suitable buffer such as 0.1 M [e.g., 0.09 M to 0.11 M] Phosphate buffered saline (PBS) or approximately 0.05 M Tris buffer at a pH of about 7.2-7.6.

For example, in some embodiments of step 104, the section of the biological sample may be incubated with a secondary antibody after incubating the section of the biological sample with a primary antibody that is specific to the antigen in which the secondary antibody is specific to the Fc portion of the primary antibody. As another example, in some embodiments of step 104, the section of the biological sample may be concurrently incubated with the secondary antibody and with a primary antibody that is specific to the antigen in which the secondary antibody is specific to the Fc portion of the primary antibody. Because of these unique characteristics the time required to stain, fluorescently tag, or otherwise label an antigen with a detectable label the tissue can be dramatically reduced by the method 100.

To better understand this reaction and the benefits of using an Fc-specific secondary antibody; let us first consider a standard immunohistochemistry staining method with a conventional anti-Ig secondary antibody. Because the secondary antibody has the capability of binding to the Fab region of the primary antibody, it cannot be incubated simultaneously with the primary antibody. If this were attempted the secondary antibody would bind to the Fab region of the primary antibody before the primary antibody had bound to its antigen. Such binding to the Fab region of the primary antibody would inhibit its binding to its antigen. In order for this system to work, the primary antibody must bind first to its antigen and this reaction must be complete prior to application of the secondary antibody. Furthermore, any excess unbound primary antibody must be rinsed off of the slide before the secondary antibody can be applied.

However, in the case of the method 100 of the present invention, this limitation may be overcome by selecting a secondary antibody that is Fc-specific. In this case the primary antibody and secondary antibody can be incubated together simultaneously, because the secondary antibody does not bind to the Fab regions and thus does not inhibit the binding of the primary antibody to its antigen. Although the secondary antibody will bind to the Fc region of the primary antibody during this incubation, this binding does not interfere with the binding of the primary antibody to its antigen. Therefore, in preferred embodiments, the method 100 may use an Fc-specific secondary antibody in step 104 so as to allow two separate conventional incubation steps to be performed as a single incubation step.

In preferred embodiments of step 104, section of the biological sample may be incubated with a secondary antibody having a detectable label using a method to rapidly bind an antigen of a biological sample with a labeled secondary antibody (“the method”) 300. In some embodiments, the method 300 may start 301 and a primary antibody mixture and a secondary antibody mixture may be selected, in which the primary antibody is specific to the antigen and in which the secondary antibody is labeled and is specific to the Fc region of the primary antibody, in step 302. Next in step 303 the primary antibody mixture and secondary antibody mixture may be applied to the biological tissue sample that may have the antigen. In some embodiments of step 303, the primary antibody mixture may first be applied to the biological sample and then the secondary antibody mixture may be applied to the biological sample while the primary antibody mixture is still in contact with the biological sample. In further embodiments of step 303, the secondary antibody mixture may first be applied to the biological sample and then the primary antibody mixture may be applied to the biological sample while the secondary antibody mixture is still in contact with the biological sample. In still further embodiments of step 303, the primary antibody mixture and the secondary antibody mixture may be simultaneously applied to the biological sample, such as by premixing the primary and secondary antibody mixtures before application. Once both the primary antibody mixture and the secondary antibody are applied to the biological sample, the method 300 may finish 304.

In step 105, unbound secondary antibody may be removed from the section of the biological sample. After a sufficient length of time has passed in step 104, such as between approximately 1 minute to 20 minutes, the tissue of the biological sample is rinsed with buffer or other suitable rinsing medium such that all unbound secondary antibodies are removed. By removing the unbound secondary antibodies, the unbound primary antibodies may also be removed. After step 105, the method 100 may proceed to step 106 or to step 107.

If the secondary antibody used in steps 104 and 105 was an enzyme labeled antibody, the method 100 may proceed to optional step 106 in which an enzyme substrate chromogen reaction may be performed by adding a suitable substrate/chromogen mixture biological sample. The resultant enzyme-substrate reaction results in a chromogen being converted from a colorless form to a colored form. Furthermore, the chromogen is insoluble and deposits as a precipitate at the site of the enzyme. This colored reaction product is visible microscopically and is indicative of a positive reaction. After completion of the immunohistochemical stain, the tissue may be further stained with a counterstain. The counterstain will stain all the tissue elements whether or not they also have an immunohistochemical stain. The counterstain is useful for studying the overall structure and morphology of the tissue as a whole. Thus, the immunohistochemical stain provides a molecular stain and the counterstain provides a morphological stain. The counterstain may be chosen such that it would have a different color from the immunohistochemical stain so that it does not obscure the stain.

There are many counterstains that can be applied to the tissue. The counterstain must have a color distinct from the immunohistochemical stain. In the case of DAB (brown) or Fast Red (Red), a blue counterstain would be appropriate. Hematoxylin provides a blue counterstain and is the most widely used counterstain in immunohistochemistry.

In step 107, the section of the biological sample may be mounted so as to be prepared for microscopic examination. Preferred mounting procedures utilize resin-based mounting medium often referred to as permanent mounting medium. These mounting mediums contain resins and plastic dissolved in an organic solvent, such as toluene or xylene. Because these mounting mediums are insoluble with water, all the water in the tissues must be removed prior to mounting. In a typical mounting procedure, and in some embodiments of step 106, the stained or fluorescently labeled tissues are dehydrated in a series of alcohols beginning with a mixture of water and alcohol and gradually moving through a series of baths with decreasing water and increasing alcohol until the slides are in a bath of 100% alcohol. Next the alcohol is gradually removed and replaced with a solvent such as xylene or toluene by moving the slide through a series of baths beginning first with a bath of alcohol and solvent and then moving through a series of baths with decreasing alcohol and increasing solvent until the slides are in 100% solvent. Finally at this stage the slides are ready for mounting. The slides are removed from the last solvent bath, and a drop of mounting medium is applied. Next a glass coverslip is overlaid the mounting medium. The mounting medium dries and glues the coverslip over the tissue. The tissues are now ready for microscopic examination using this conventional mounting procedure. However, this conventional mounting procedure is time intensive and can take upwards of 20 minutes to complete.

In preferred embodiments, step 107 may be performed using a method of rapidly dehydrating a biological sample (“the method”) 400. The method 400 may be used as a rapid and simplified method to dehydrate the tissues to prepare the biological sample for mounting after unbound secondary antibody is removed from the section of the biological sample. The method 400 may employ a mixture of an alcohol and an alcohol soluble polymer or plastic, such as 10% polyvinyl in ethanol. Additionally, the alcohol soluble polymer must also be soluble in water. Example alcohol and water soluble polymers include: Polymers of vinyl, such as polyvinyl alcohol, polyvinyl acetate, polyvinyl pyrrolidone, and polyvinyl butyral; Polymers of ethylene, such as polyethylene glycol; and Polymers of cellulose such as, ethyl cellulose, propyl cellulose, hydroxypropyl cellulose, hydroxymethyl cellulose, and hydroxypropyl methylcellulose.

In some embodiments, the method 400 may start 401 and a mixture of an alcohol and an alcohol and water soluble polymer, the mixture comprising an alcohol and plastic dehydrating reagent, may be applied to a section of a biological sample in step 402. Optionally, a mixture of the alcohol and plastic is prepared as needed for dehydration. In preferred embodiments, the alcohol and plastic dehydrating reagent is applied to the slide holding the biological sample such that any remaining water is rapidly displaced by the dehydrating reagent (generally by applying an excess of the alcohol and plastic dehydrating reagent). Because the plastic is also water soluble, it will not precipitate when exposed to the remaining water on the slide. In further preferred embodiments, and when used in the performance of a method for rapid immunohistochemical detection of an antigen from a biological sample 100, step 402 may be performed after counterstained slides are rinsed with water to remove excess counterstain, unbound antibody, etc.

In step 403, the alcohol of the alcohol and plastic dehydrating reagent is allowed to dry, such as by air drying. The air drying process takes approximately 10-20 seconds to complete because the alcohol dries very rapidly. Furthermore, the slide dries with a thin layer of the plastic embedded into the tissue and overlaying the tissue. The plastic provides structure and support to the tissue as it dries and prevents drying artifacts, such as tissue shrinkage or cracking that would occur in the absence of the plastic. Furthermore, the plastic layer can act as a barrier between the tissue and the mounting medium. This can act to protect chromogens from fading when in direct contact with the mounting medium. After step 403, the method 400 may finish 405 or the method 400 may proceed to optional step 404 so that the method 400 may function as a method of rapidly dehydrating and mounting a biological sample.

In optional step 404, mounting medium may be applied to the biological sample. In preferred embodiments, after the alcohol of step 403 applied to the biological sample slide is dried a drop of resin-based mounting medium, often referred to as permanent mounting medium, may be added on the biological sample and then overlaid with a glass coverslip. The entire process of the method 400, including optional step 404 that preferably uses a resin-based mounting medium, takes less than approximately 30 seconds. In further embodiments, step 404 may be performed using an aqueous mounting medium that is composed of one or more water-soluble plastics and polymers. This type of mounting medium can be added directly onto a wet microscope slide without the requirement for first dehydrating the slide. Although aqueous mounting is convenient, the optical resolution for aqueous mounting media are considered inferior to permanent mounting media. Thus, for best resolution resin-based (permanent) mounting media are preferred. After step 404, the method 400 may finish 405.

After step 107, the method 100 may continue to step 108 of detecting secondary antibody bound to the section of the biological sample, preferably via microscopic examination, which may be the final step in the immunohistochemistry detection process and the method 100 may finish 109. This step of detecting secondary antibody bound to the section of the biological sample is the same whether a conventional immunohistochemistry method or whether a rapid immunohistochemistry method 100 is used. However, the rapid immunohistochemistry method 100 is uniquely applicable to certain situations where rapid diagnosis is required. One such application is Moh's micrographic surgery, for example in the examination of skin cancers. This method requires excision of the suspected lesion, staining, and microscopic examination. In this examination the primary goal is to examine the surgical margins for the presence or absence of tumor cells. If tumor cells are present at the surgical margin, then the surgeon will remove additional tissue until the surgical margins are clear of tumor cells. Thus, the patient must remain for long periods of time while the tissues are stained and inspected. Typical stains used for this procedure include Hematoxylin and eosin (H&E) because they provide rapid results. However, H&E only provide morphological information and do not provide molecular information. Furthermore, a few tumor cells present near the surgical margins could be missed by this method of staining. Immunohistochemical staining could improve on this sensitivity but is rarely used because the long times associated with producing a stain. Thus, immunohistochemical stains are considered incompatible with this method. As can be seen by this brief description there is a need for a rapid immunohistochemical stain which could provide results in approximately the same time frame as a conventional H&E stain. The method 100 of the present invention is able to achieve this goal.

In step 108, after mounting the stained slides are ready for microscopic review. When viewed microscopically the tissues will typically show an abundance of counterstained cells, blue in the case of Hematoxylin. The microscopist will be able to discern certain morphological features such as tissue type and the presence or absence of tumor material. However, toward the margins of the tumor the identification of individual tumor cells becomes more difficult as they may be obscured by the more abundant normal cells. It is critical to identify all tumor cells at the margins of the surgical incision. If tumor cells are identified up to the surgical margins it is likely that some tumor cells remain in the body and further surgery is indicated.

The use of immunohistochemical staining methods provides increased sensitivity for detecting isolated tumor cells, particularly at the surgical margins. These isolated tumor cells will stain according to the chromogen used (typically brown or red) and are easily identified, even among an abundance of normal cells. Thus, the identification of colored reaction product is indicative of a positively-staining tumor cell. Despite this apparent improvement in sensitivity, immunohistochemistry is rarely used because the length of time required to process slides by immunohistochemistry is incompatible with the rapid staining requirements of the Moh's procedure.

### Connections of Main Elements and Sub-Elements of Invention

The IHC staining method 100 has been developed specifically for rapid immunohistochemical staining. As such each of the steps has been optimized for speed. Although some of the steps that have been incorporated into this staining method utilize conventional histochemical or immunohistochemical techniques, many of the steps have been modified to accomplish staining within a 10 minute time frame. These steps include:

Step 102—depositing a section of the biological sample on a slide. In preferred embodiments, step 102 may comprise preparation of a biological sample by flash freezing and cutting a thin section of sample from the frozen block and affixing the sections onto a microscope slide.

Step 103—permeabilizing the section of the biological sample. In preferred embodiments, step 103 may comprise permeabilizing the tissue sample to expose hidden antigens via a permeabilization reagent that is heated to between approximately 25 degrees and 95 degrees Celsius. In further preferred embodiments, step 103 may comprise permeabilizing the tissue sample to expose hidden antigens via a permeabilization reagent that is heated to approximately 90 degrees Celsius for approximately one minute.

Step 104—incubating the section of the biological tissue sample with secondary antibody having a detectable label. In preferred embodiments, step 104 may comprise one or more of: staining the tissue with a primary antibody; staining or labeling the tissue with a secondary antibody either simultaneously with the primary antibody or sequentially without rinsing the primary antibody; staining or labeling with a secondary antibody that is Fc-specific with respect to the primary antibody; staining or labeling with a secondary antibody that is a polymer; staining or labeling with a secondary antibody that has a detectable enzyme attached; staining or labeling with a secondary antibody that has a fluorescent tag attached; and staining or labeling with a substrate/chromogen that is specific to the detectable enzyme, thereby producing an insoluble colored reaction product at the site of the enzyme.

Step 105—removing unbound secondary antibody from the section of the biological sample, preferably via rinsing with water or other suitable rinsing medium.

Optional Step 106—performing enzyme substrate chromogen reaction, such as for counterstaining the tissue.

Step 107—mounting the section of the biological sample. In preferred embodiments, step 107 may comprise rapidly dehydrating the tissue and simultaneously embedding the tissue with application of a transparent alcohol and a water and alcohol soluble plastic. In further preferred embodiments, step 107 may comprise mounting the dehydrated tissue with a resin-based mounting medium.

Step 108—detecting secondary antibody bound to the section of the biological sample, preferably via observing the presence or absence of colored chromogen with a microscope or other visualization tool.

### Alternative Embodiments of Invention

An object of the present invention is to provide a method to stain tissues using

immunohistochemistry such that the entire staining method can be completed in about 10 minutes. Other methods using immunohistochemistry have been described. However, these methods typically use standard immunohistochemical staining methods and reduce incubation times for each step in order to achieve rapid results. However, these methods have, for the most part, produced weak staining with low sensitivity. Such low sensitivity does not provide any added value over a conventional H&E stain, and these methods are rarely used in clinical practice.

For difficult cases, where either the diagnosis is uncertain or there are remaining questions regarding the surgical margins, a standard immunohistochemistry stain may be required. However, in these cases the stains are usually not completed until several days later. If additional finding are uncovered the patient may be recalled for additional surgery. The present invention of rapid immunohistochemical staining is able to overcome these limitations.

In preferred embodiments, the present invention may comprise a Rapid stain for IHC that can be used concurrently with Mohs surgery and standard Mohs procedures.

### Application Of The First Stain

The standard Mohs staining procedure is comprised of two stains, a first stain of hematoxylin and a second stain of eosin. With respect to the first stain of hematoxylin, the nuclei of each cell are stained blue. A cancer cell can be identified by the large pale blue staining of the nucleus, areas of condensed chromatin (dark blue), and possibly the presence of nucleoli (round blue). This morphology is distinct from the morphology of the more numerous normal skin cells such that a trained microscopist can distinguish between tumor cells and normal cells. Next, and a second stain of eosin is applied. The eosin imparts a reddish-pink color to the cytoplasm of the cells. The finished slides are then evaluated microscopically where each cell will have various shades of blue and red, and the microscopist will attempt to identify tumor cells by virtue of their morphological characteristics.

Typically, this evaluation would be conducted by an expert pathologist who has years of training in the morphological identification of different cells types. However, even for an expert pathologist the presence of rare tumor cells may be missed because they become obscured by the more numerous normal cells or the tumor cells may have been cut through when the skin sample was prepared. Usually in such cases the examination of the skin sample may produce equivocal results in which case the remaining sample may be submitted for additional IHC staining. In the staining method of the present invention, the first stain is Celestine Blue rather than hematoxylin. Celestine Blue has certain advantages when used as a first stain. Its color and staining characteristic have been found to be similar or identical to hematoxylin. However, it is much more stable than hematoxylin and retains its staining characteristic even when exposed to harsh methods, such as subsequent staining procedures. The present invention shows that Celestine Blue can be applied to a tissue as a first stain and will retain its staining characteristic even when a second staining method such as IHC is applied. In the standard Mohs procedure if the presence of rare tumor cells were missed, this would result in a misdiagnosis. Such a misdiagnosis could lead to a false negative conclusion where it is believed that no tumor cells were present, when in fact rare tumor cells were still present. Usually in such cases where the results are equivocal the skin sample may be further processed by use of an IHC stain. Typically, the sample would be sent to a specialized histology lab for overnight processing. However, if one were to link the Mohs surgery to immediate IHC several modifications would be required. First, the stain of hematoxylin would require changing. In standard IHC methods the counterstain, such as hematoxylin, would be applied as the last step after the IHC stain. This would ensure that the hematoxylin would not fade or be washed away by the IHC staining procedure. However, when combining IHC with the Mohs procedure the first blue stain would be applied before the IHC stain. This provides the microscopist with the opportunity to evaluate the slide prior to deciding as to whether to proceed with IHC or just complete the Mohs staining procedure. In the event that the decision is to proceed with IHC the first blue stain needs to be sufficiently stable that it will not fade or be washed away with subsequent IHC staining. Thus, hematoxylin is unsuitable for this purpose.

On the other hand, Celestine Blue can be substituted for hematoxylin. Celestine Blue stains virtually identically with hematoxylin, but has the advantage of greater stability and is not washed away be the subsequent steps of IHC staining. Thus the blue stain of Celestine Blue can be applied to the tissue prior to IHC staining. Once the IHC staining is completed, no further counterstaining is required thus saving additional time to completion.

A useful example method for preparing Celestine Blue for staining is:

Celestine Blue at 3-5% (w/v), preferably 4% (w/v)

Dimethylformamide at 9-11% (v/v), preferably 10% (v/v)

Glycerol at 9-11% (v/v), preferably 10% (v/v)

Ferric ammonium sulfate at 1-3% (w/v), preferably 2% (w/v)

These components are prepared in a water-based solution. Slides are stained in the Celestine Blue solution for about 30 seconds (plus or minus 10 seconds).

The advantage of this staining method will become clear as described in the following disclosure and examples.

The method of the present invention requires that the blue counterstain be applied before the IHC stain. This sequence of steps is the opposite of how IHC staining is typically performed where the blue counterstain is applied as the last step of the sequence. When applying a counterstain as the first step certain qualities of the counterstain are required:
1. It must be stable and unaffected by the subsequent steps of IHC.
2. It must provide staining characteristics identical to the standard hematoxylin.
3. It must not interfere with the subsequent steps of IHC. In particular it must not interfere with the binding of antibodies to their targets.

### Application Of A Second Stain.

The second stain to be applied will be dependent on the evaluation of the first stain. In some instances, the second stain will be the standard Mohs second stain of eosin. In other instances, the second stain will be an IHC stain. The choice of the second stain will be dependent upon the expert analysis of the microscopist. Therefore, at this step a decision point is reached.

Following application of a first stain of Celestine Blue, the stained slide can be evaluated microscopically, even before the application of a second stain. This is because the Celestine Blue is sufficient in most cases to identify the presence of tumor cells. At this point the microscopic evaluation may reveal the unequivocal presence of tumor cells in which case additional Mohs surgery would be required. Under these circumstances there would be no need to proceed to IHC. The decision would be to finish the staining with the second stain of eosin, and then proceed with additional surgery. On the other hand, if the examination of the slide failed to identify the presence of tumor cells or if the results were equivocal, then the decision point would favor proceeding with the IHC stain as the second stain. The Mohs surgical procedure and staining would be improved by providing a decision point after the first stain as to whether to conclude the staining procedure with eosin or whether to proceed with an IHC stain.

In order for IHC staining to be a viable option to be performed in conjunction with Mohs staining a new method of rapid IHC staining is required. Conventional IHC methods are too laborious and time consuming to be used in conjunction with Mohs surgery. In most cases IHC would not be performed even if it could provide additional confirmatory results. This could result in occasional false negative results being reported. In cases where IHC staining was performed, this would require several days until results were available. A positive result at this stage would require recalling the patient for additional surgery. The process could be improved with a rapid method of IHC that could be performed concurrently with Mohs surgery.

### Rapid IHC Staining As A Second Stain

A decision to proceed with IHC staining could have a major benefit to the Mohs staining method if the IHC staining method could be improved to provide rapid results. This would allow the IHC staining method to be used while the patient was still in surgery.

If an IHC staining method could reveal the presence of residual tumor cells that would have been missed by standard Mohs staining methods, additional surgery could be performed while the patient was still present and already prepared for surgery, thus negating the necessity for recalling the patient for additional follow-up surgery. In order to achieve this goal several modifications of the Mohs and IHC staining methods would be needed. Changes in the Mohs staining procedure include:
1. Substituting Celestine Blue for hematoxylin as a first stain.
2. Evaluating a first stain and deciding as to whether to proceed with a second stain of eosin or proceed with a second IHC stain.

### Modifications of the IHC Procedure Include:

1. A first stain of Celestine Blue applied before the IHC stain.
2. A modified rapid IHC staining method that provides results within a few minutes, such that the IHC stain can be performed while the patient is still in surgery.

### Details for Rapid Immunohistochemical Staining Method

Preparing A Biological Sample. Freezing And Sectioning

For rapid IHC it may be preferable to freeze the tissue rather than embed the tissue in paraffin because the time required to process a frozen tissue is significantly shorter than the time required to embed a tissue in paraffin. The frozen tissue can be flash frozen in liquid nitrogen or a low temperature bath of iso-pentane and dry ice. The solid frozen tissue can then be embedded into a block and attached to the chuck of a cryostat using a suitable embedding medium, such as OCT. The cryostat is a device that can cut thin sections from the frozen tissue while maintaining a sub-zero temperature, such as −20 C. The tissue is typically sectioned at about 4-8 u to produce very thin tissue sections. The tissue sections are then place onto a cold microscope slide where they become adherent. The microscope slides with adherent tissue are ready for pre-treatment in preparation for IHC staining.

### Permeabilization

The microscope slide with the attached tissue is brought to room temperature, at which time the tissue melts and attaches to the microscope slide. Based on the molecular structure of the tissue and the specific antigens under investigation, the antigens may be hidden or otherwise inaccessible to the primary antibody. Therefore, it has been found to be useful to first permeabilize the tissue prior to staining. Although the exact mechanism by which the permeabilization reagent exposes hidden antigens is unknown, it is thought to act by changing the molecular structure of the tissue and antigens.

The permeabilization reagent preferably is approximately a 0.05 M Tris buffer containing about 5-10 mg/ml of ethylenediaminetetraacetic acid (EDTA) and about 1% detergent (plus or minus 0.5%) such as Tween 20. The detergent in the permeabilization reagent may destroy or dissolve lipid layers and other hydrophobic molecules thereby allowing the hydrophilic antibodies to more easily penetrate the tissues. Furthermore, chelating agents may be useful in helping to stabilize proteins.

The slides with attached tissues are submerged into the permeabilization reagent and incubated for a sufficient length of time that the tissues are permeabilized. The length of time necessary for permeabilization varies depending on the temperature of the incubation mixture. We have found that a permeabilization reagent at about 90 C will permeabilize the tissue in about one minute. Because this short incubation time is compatible with rapid IHC we have chosen this as the preferred temperature. Furthermore, because of the short incubation time, the entire permeabilization step can be conducted at room temperature with only slight cooling of the permeabilization reagent. After about 1 minute (plus or minus 10 seconds) in the permeabilization reagent the slides are removed and rinsed with a 0.05 M Tris buffer in preparation for IHC staining.

Permeabilization can also be conducted at different temperature, but the incubation times may have to be increased to achieve sufficient permeabilization. For example, we have discovered that sufficient permeabilization will occur even under room temperature incubation conditions if the incubation time is extended to 60 minutes.

### Immunohistochemical Staining: Primary Antibody

The first step in the IHC staining process is the application of a primary antibody onto the tissue. If the antigen of interest is present in the tissue, then the primary antibody will bind to its antigen. On the other hand, if the antigen of interest is not present, then no binding event will occur.

For example, in the diagnosis of a carcinoma an antibody to a carcinoma antigen, cytokeratin for example, may be applied to the tissue. If the tissue contains the cytokeratin antigen, then the antibody will bind to the tissue, but if no cytokeratin is present then the antibody will not bind. In this example, the binding of a primary antibody to cytokeratin is indicative of the presence of a carcinoma.

Other cytokeratin positive elements may also be present, such as epithelium and sweat glands. However, these normal elements can be distinguished from carcinoma based upon their distinct morphology.

Following permeabilization the tissues are ready for IHC staining. For all IHC staining steps an appropriate buffer must be used. The typical buffer would be similar to physiological conditions in terms of molarity and pH. These conditions favor the binding of antibodies to antigens and enzyme-substrate reactions.

The first step in the staining process is to apply the primary antibody in a suitable buffer solution. The antibody is selected to bind to the antigen of interest, where the antigen of interest may be a protein that helps identify the particular tissue or tumor under investigation. If the primary antibody binds, then the antigen of interest is present, whereas if the primary antibody does not bind, then the antigen of interest is absent. A positive binding event will allow identification of the tumor cells. The primary antibody is applied to the tissue and incubated for a sufficient length of time to allow binding to occur.

The primary antibody is typically generated by immunizing an animal with the antigen of interest. Commonly primary antibodies are produced in mice and in rabbits, although other animals can also be used. Typically the antibodies are monoclonal antibodies, where the antibody is generated from a single clone of antibody-producing cells, or the antibodies may be polyclonal where the antibodies are produced from multiple different antibody-producing cells. A polyclonal antibody may be harvested from the serum of the immunized animal and in this state may be referred to as antiserum.

Immunohistochemical Staining: Fc-Specific Secondary Antibody-Enzyme Conjugate

The second step in this rapid IHC process is to determine whether a binding event of primary antibody to antigen has occurred. In order to make this determination a secondary antibody is next applied. The secondary antibody has several unique features that allow it to be used in this respect. First the secondary antibody must be specific for the first primary antibody. For example, if the primary antibody has been produced in a mouse, then the secondary antibody must be anti-mouse IgG specific. If the primary antibody has been produced in a rabbit then the secondary antibody must by anti-rabbit IgG specific. The second feature of the secondary antibody is that it must have a detectable tag such as an enzyme. Commonly used enzymes in immunohistochemistry include peroxidase and alkaline phosphatase. Furthermore, then enzymes may be directly attached to the secondary antibody or attached via a polymer backbone. The advantage of using a polymer backbone is that multiple different enzyme molecules and multiple different secondary antibodies can be contained within a polymerized molecular structure, thus providing increased sensitivity to the detection method. The third, and unique aspect of this invention, is that the secondary antibody must be Fc-specific. Fc-specific antibodies have unique specificity in that they bind to only a certain region of the primary antibody and this region is termed the Fc portion. The Fc portion of an antibody is not involved in the binding of an antibody to its antigen. The antibody combining regions are termed Fab. Therefore, at one end of the antibody molecule are the Fab regions and on the other end of the antibody molecule are the Fc regions. A secondary antibody that is not Fc-specific will bind to all regions of the primary antibody including the Fab and Fc regions.

In conventional immunohistochemistry the primary antibody is incubated with the tissue for a sufficient length of time in order for the antibody to bind to its epitope. After a sufficient length of time has passed the tissue is rinsed such that all unbound antibodies are removed. Any bound antibodies will remain attached to the tissue. Next a secondary antibody is applied and incubated for a sufficient length of time that it will bind to its primary antibody, if present. However, in the present invention the primary antibody and the secondary antibody can be incubated together at the same time, or the secondary antibody can be added to the slide at any point after the primary antibody without the requirement to first rinse off unbound primary antibody. Because of these unique characteristics the time required to stain the tissue can be dramatically reduced.

To better understand this reaction and the benefits of using an Fc-specific secondary antibody, let us first consider a standard IHC staining method with a conventional anti-IgG secondary antibody. Because the secondary antibody has the capability of binding to the Fab region of the primary antibody, it cannot be incubated simultaneously with the primary antibody. If this were attempted the secondary antibody would bind to the Fab region of the primary antibody before the primary antibody had bound to its antigen. Such binding to the Fab region of the primary antibody would inhibit its binding to its antigen. In order for this system to work, the primary antibody must bind first to its antigen and this reaction must be complete prior to application of the secondary antibody. Furthermore, an excess unbound primary antibody must be rinsed off of the slide before the secondary antibody can be applied.

However, in the case of the present invention we have overcome this limitation by selecting a secondary antibody that is Fc-specific. In this case the primary antibody and secondary antibody can be incubated together simultaneously, because the secondary antibody does not bind to the Fab regions and thus does not inhibit the binding of the primary antibody to its antigen. Although the secondary antibody will bind to the Fc region of the primary antibody during this incubation, this binding does not interfere with the binding of the primary antibody to its antigen. Therefore, by using an Fc-specific secondary antibody we are able to combine two separate incubation steps into a single incubation step. This dramatically reduces the total time required to complete the IHC stain.

The incubation of the primary antibody and secondary antibody can be performed in a variety of ways. The primary antibody and secondary antibody can be premixed, and then applied to the tissue as a single reagent. The primary antibody can be applied to the tissue and allowed to react for a short period of time, and then without rinsing the secondary antibody can be directly added to the incubation mixture. And finally the primary antibody can be incubated for a short period of time, the excess reagent drained from the slide without rinsing, and then the secondary antibody added to the remaining reactants.

In the case where the primary antibody and Fc-specific secondary antibody are premixed before adding to the tissue, the primary antibody would typically be used at a concentration of about 2-5 ug/ml, and the Fc-specific secondary antibody would be used at a concentration of about 5-15 ug/ml. As a general rule the ratio of the primary antibody to Fc-specific secondary antibody would be about 1:3 weight by weight (w/w) [e.g., 1:2.5 to 1:3.5 (w/w)]. The complex of primary and secondary antibody is prepared in a suitable buffer such as 0.1 M [e.g., 0.09 M to 0.11 M] Phosphate buffered saline (PBS) or approximately 0.05 M Tris buffer at a pH of about 7.2-7.6.

### Immunohistochemical Staining: Detection

After binding of the secondary antibody to the primary antibody the complex now contains a detectable label such as an enzyme. Different enzymes can be used in IHC including beta-galactosidase, peroxidase, and alkaline phosphatase. Peroxidase derived from horseradish is the most commonly used enzyme. There are several substrate/chromogens that are used with horseradish peroxidase, the most common of which is diaminobenzidine (DAB). DAB produces a brown precipitate in the presence of horseradish peroxidase. Thus the presence of a brown stain on the tissue is indicative of a positive reaction. Alkaline-phosphate is less commonly used, and there are a number of different substrate/chromogens that can be used with alkaline phosphatase. The most commonly used chromogen is Fast Red that produces a red reaction product at the site of the enzyme. Thus, the presence of a red stain on the tissue is indicative of a positive reaction. The resultant enzyme-substrate reaction results in a chromogen being converted from a colorless form to a colored form. Furthermore, the chromogen is insoluble and deposits as a precipitate at the site of the enzyme. This colored reaction product is visible microscopically and is indicative of a positive reaction.

### Dehydration and Sealing

Having completed the IHC stain, the final steps include preparing the slides for microscopic examination. Although a counterstain would generally be applied at this point, the present invention does not require a counterstain as this has already been applied prior to the IHC stain. Additional time has been saved by avoiding the counterstain at this point. A counterstain will stain all tissue elements such that the overall structure and morphology of the tissue will be apparent when viewed microscopically. The counterstain of Celestine Blue will stain nuclei blue and provide excellent contrast against commonly used chromogens such as DAB (brown) and Fast Red (red). In this example a negative tissue would have only blue staining cells, whereas a positive staining tissue would contain both blue and brown (DAB) or blue and red (Fast Red) staining cells.

The tissue is now ready for mounting. Preferred mounting procedures utilize resin-based or permanent mounting medium. These mounting mediums contain resins and plastics dissolved in an organic solvent such as toluene or xylene. Because these mounting mediums are insoluble with water, all the water in the tissues must be removed prior to mounting. In a typical mounting procedure, the stained tissues are dehydrated in a series of alcohols beginning with a mixture of water and alcohol and gradually moving through a series of baths with decreasing water and increasing alcohol until the slides are in a bath of 100% alcohol. Next the alcohol is gradually removed and replaced with a solvent such as xylene by moving the slide through a series of baths beginning first with a bath of alcohol and xylene and then moving through a series of baths with decreasing alcohol and increasing xylene until the slides are in 100% xylene. Finally at this stage the slides are ready for mounting. The slides are removed from the last xylene bath, and a drop of mounting medium is applied. Next a glass coverslip is overlaid the mounting medium. The mounting medium dries and glues the coverslip over the tissue. The tissues are now ready for microscopic examination.

As can be seen by this description there is a need for a rapid and simplified method to dehydrate the tissues and prepare them for mounting. The present invention describes a method and a reagent to accomplish rapid dehydration. The method employs a mixture of an alcohol and an alcohol soluble polymer or plastic. Additionally, the polymer must also be soluble in water. In this method a solution of the alcohol and plastic is prepared for dehydration. After staining the slides are rinsed with water. Next the alcohol and plastic dehydrating reagent is applied to the slide such that any remaining water is rapidly displaced by the dehydrating reagent. Because the plastic is also water soluble it will not precipitate when exposed to the remaining water on the slide. After the water is expelled, the slides are allowed to air-dry. The entire process takes about 10-20 seconds to complete because the alcohol dries very rapidly. Furthermore, the slide dries with a thin layer of plastic embedded into the tissue and overlaying the tissue. The plastic provides structure and support to the tissue as it dries and prevents drying artifacts, such as tissue shrinkage that would occur in the absence of the plastic. Furthermore, the plastic layer can act as a barrier between the tissue and the mounting medium. This can act to protect chromogens from fading when in direct contact with the mounting medium. Finally, after the slides are dried a drop of mounting medium can be added and then overlayed with a glass coverslip. Then entire process of rapid dehydration and mounting takes less than 30 seconds.

Another method of mounting stained slides is with an aqueous mounting medium. In this method the mounting medium is composed of water-soluble plastics and polymers. This type of mounting medium can be added directly onto a wet microscope slide without the requirement for first dehydrating the slide. Although aqueous mounting is convenient the optical resolution is inferior to permanent mounting media. Thus, for best resolution resin-based (permanent) mounting media are preferred.

### Mounting And Microscopic Analysis

The final step in the IHC staining process is microscopic examination. This step is the same whether a conventional or rapid IHC method is used. However, our rapid IHC method is uniquely applicable to certain situations where rapid diagnosis is required, such as Mohs micrographic surgery. The use of an IHC staining method provides increased sensitivity for detecting isolated tumor cells, particularly at the surgical margins. These isolated tumor cells will stain according to the chromogen used (typically brown or red) and are easily identified, even among an abundance of normal cells. Thus, the identification of colored reaction product is indicative of a positively-staining tumor cell.

As can be seen by this description, there is a need for a rapid IHC stain which could provide results in approximately the same time frame as a conventional H&E stain. The present invention describes a method that can achieve this goal.

Conventional IHC staining methods can be used as an alternative to the rapid IHC staining method but this requires several hours or days to complete. The rapid IHC staining method can be completed in less than 20 minutes.

The rapid IHC staining method achieves rapid staining by modifying standard IHC by the following steps:
1. Tissues are subjected to a permeabilization step that allows rapid penetration of antibodies into the tissue.
2. Incubation of a primary antibody complex comprised of a structure containing 1) primary antibody, 2) Fc-specific secondary antibody with specificity for the Fc region of the primary antibody, 3) enzyme(s).
3. A colorless chromogenic substrate that is converted to a colored reaction product by enzymatic activity.
4. A microscopic evaluation of the stained slide to determine the presence or absence of the colored reaction product. The presence of such reactive product is indicative of a positive test.

### Connections of Main Elements and Sub-Elements of Invention

The present invention provides methods for integration of a standard Mohs surgical procedure and tissue evaluation with a rapid method of IHC staining. The advantage of this integration is that it provides a more sensitive method of evaluating surgical samples at the time of initial surgery. If additional surgery becomes necessary to remove all residual cancer cells, this can be performed immediately and does not require recalling the patient for follow-up surgery. The procedure for integration is as follows.
1. Patient undergoes Mohs surgery for a suspected skin cancer.
2. The surgical sample is snap frozen and sectioned into thin slices using a cryostat.
3. Thin sections are then applied to microscope slides for staining.
4. The microscope slide with the attached section is prepared for staining by treatment with a permeabilization agent. The permeabilization reagent conditions the tissue to allow the rapid penetration of the tissue with subsequent IHC reagents.
5. The tissue is stained with a first stain of Celestine Blue which stains the nucleus of all cells.
6. The microscopist evaluates the stained tissue looking for the morphological patterns of tumor cells.
7. A decision point is reached as to whether to proceed with a standard Mohs stain using eosin as the second stain, or whether to proceed with a more sensitive IHC stain.
8. If no tumor cells are observed with the first stain, or if the first stain yields equivocal results, then the decision point will favor a second stain using IHC staining methods.
9. The IHC staining method is a rapid IHC staining method which can be completed in less than 10 minutes. The steps of the rapid IHC staining method include:
A. Incubate slide and tissue with primary antibody. The primary antibody is selected based on its specificity and ability to recognize and bind to target molecules expressed by the suspected tumor cells. The primary antibody has been prepared to facilitate rapid IHC by reacting the primary antibody with a secondary primary antibody. The secondary antibody has the following characteristics: 1) is an Fc-specific secondary antibody with the ability to react specifically with the Fc portion of the primary antibody, 2) is conjugated to an enzyme molecule. The mixture of the primary antibody and the secondary antibody complex, create a new complex molecule composed of primary antibody-secondary antibody-enzyme. The resulting complex when incubated with the tissue will bind to its molecular target thereby incorporating enzymes onto the molecular target. All of this complex reaction can occur very rapidly with a single incubation step.
B. The bound enzyme is then reacted with a colorless chromogen which is converted via enzymatic activity into a colored reaction product.
C. The colored reaction product can be viewed microscopically to reveal the

presence of rare tumor cells. The presence of colored cells confirms the presence of tumor cells, whereas the lack of colored cells confirms the absence of tumor cells.

### Operation of a Preferred Embodiment

This invention discloses methods for staining and evaluating tissue samples obtained by Mohs micrographic surgery. The Mohs method is intended for the diagnosis and surgical treatment of presumptive skin tumors, such as squamous cell carcinoma, basal cell carcinoma, and malignant melanoma. In the Mohs procedure skin layers containing the suspected cancer are surgically removed, stained, and examined microscopically to determine the type of cancer and the extent of invasion. If cancer cells are present in the excised tissue, then another layer of skin is removed and examined. This procedure is repeated until the excised tissue shows that no tumor cells are present. The procedure is designed to be rapid so that successive layers of skin can be removed and examined during a single operation. If successful the patient will not require any further surgeries. However, if the microscopic examination fails to identify rare residual tumor cells, then recurrence of the tumor is likely, thus necessitating additional surgeries at a future time.

The sensitivity of the procedure for detecting rare tumor cells could be improved by the use of IHC stains. This method of staining relies upon the detection of certain molecular markers which can be used to identify not only the presence of tumor cells but also the specific type of cancer. While standard Mohs stains rely exclusively on morphology of the tumor cells and the expertise of the microscopist, IHC methods do not rely solely on morphology but primarily rely upon the positive identification of molecular targets. This feature makes IHC stains more reliable for detection of rare cancer cells compared to conventional Mohs H&E stains.

The Mohs staining method could be improved by incorporating IHC stains in the procedure. However, this is rarely done because IHC stains require several hours or days to complete. Thus, IHC staining is incompatible with the rapid Mohs procedure which can be completed in minutes rather than hours.

In the standard Mohs method the surgical sample is first frozen and is then sectioned by a cryostat. Each thin tissue section is then adhered to a microscope slide in preparation for staining. In the standard Mohs staining procedure hematoylin is first applied followed next by eosin to produce the standard H&E stain. The stained slides are then examined microscopically to determine whether the surgery has been successful in removing all cancer cells or whether additional surgery is required. The present invention improves upon the standard Mohs procedure in several important respects. First, after the tissue sections on microscope slides have been prepared, the slides are subjected to a brief incubation in a permeabilization reagent in preparation for potential IHC staining. Next, the tissues are incubated with a first stain of Celestine Blue rather than hematoxylin. While similar to hematoylin in appearance and performance Celestine Blue stain is much more stable when slides are subjected to further staining methods such as IHC stains. Next the slides are examined for the presence of tumor cells. A critical step in our improved Mohs staining method is the decision point. At this juncture the surgeon has two options. If presumptive tumor cells are observed after the first stain the surgeon will opt to finish the stain with eosin for a final definitive identification and then proceed to further surgery. If no tumor cells are observed after the first stain, or if staining results are equivocal, the surgeon will opt to perform the more sensitive IHC stain.

In order for the IHC stain to be useful at this juncture, the time-intensive IHC staining method must be shortened from several hours to just a few minutes. By performing a rapid IHC method, the slides can be stained, evaluated, and if necessary additional surgery can be performed while the patient is still present and on the operating table.

The rapid IHC staining method can be accomplished by a number of changes from the standard IHC staining method. First, a permeabilization reagent is applied to facilitate the rapid movement of antibodies and reagents into the tissue. Next a conjugate is applied that contains both primary antibody-secondary antibody-enzyme complexes. These complexes are formed ahead of time by mixing together 1) a primary antibody, 2) an Fc-specific secondary antibody with specificity for the Fc region of the primary antibody and that has also been conjugated to multiple enzyme molecules. This conjugate combines the individual steps of sequential application of primary antibody and then secondary antibody as performed in standard IHC methods. The slides are then stained with a colorless chromogen that, when reacted with the enzyme, produces a colored reaction product that is deposited at the site of the target molecules, or in this case the cancer cells. Microscopic examination of the IHC stained slides can then reveal the presence of absence of rare tumor cells. IHC staining is capable of detecting a single cancer cell among several hundred normal cells.

### OPERATION OF PREFERRED EMBODIMENT EXAMPLES

The following Examples are shown to further illustrate the present Invention.

### Example 1

### Tissue Preparation

Moh's micrographic surgery was used to obtain a skin biopsy containing a

presumptive squamous cell carcinoma. The biopsy was frozen in an isopentane and dry ice bath. The frozen tissue was sectioned on a cryostat at −20 C at thickness of 6 micrometers (u), and attached to a pre-cooled silanized microscope slide. The slides with the attached tissues where then either dried at room temperature (air-dried) or sprayed with a cytological spray fixative composed of polyethylene glycol and ethanol and then dried at room temperature (fixed). Once dried the slides were either stained immediately or stored at −20 C prior to staining.

The purpose of the following experiment was to determine the best method for preparation and storage of the slides. Although storage of unstained slides is not a routine procedure in a clinical setting, for research purposes the ability to store unstained slides is advantageous in that it provides a consistent source of unstained materials over the course of these studies. Slides were stained by immunohistochemistry with the antibody to cytokeratin, clones AE1 and AE3 to demonstrate squamous cell carcinoma as follows:
1. AE1/AE3 primary antibody incubation for 20 minutes.
2. Anti-mouse HRP-Polymer incubation for 20 minutes.
3. DAB incubation for 5 minutes.
4. Counterstain with Hematoxylin for 3 minutes.
5. Mount and view with a microscope.
6. Brown staining due to DAB chromogen was scored on a semi-quantitative scale of 0 to 3 as follows:
A. 0 =no staining.
B. 0.5=staining less than 1
C. 1 =weak staining.
D. 1.5 =staining greater than 1 but less than 2.
E. 2 =moderate staining.
F. 2.5 =staining greater than 2 but less than 3
G. 3 =strong staining.

TABLE 1
Results for Example 1. Tissue Preparation.

| Specimen | | Scoring for | |
|---|---|---|---|
| Type | | AE1/AE3 | Observations |
| Fresh air-dried | | 2.0 | |
| Fresh Fixed | | 2.5 | better morphology than air-dried |
| 3 | day air-dried | 2.0 | |
| 7 | day air-dried | 2.0 | |
| 21 | day air-dried | 1.5 | |
| 3 | Fixed | 2.5 | better morphology than air-dried |
| 7 | Fixed | 2.5 | better morphology than air-dried |
| 21 | Fixed | 2.0 | better morphology than air-dried |

These results showed that both fresh and stored tissue samples were equivalent up to day 7. By day 21 there was only a slight decrease in staining. Furthermore, these studies showed that both air-dried and fixed tissue samples could be used. In general, the fixed tissue samples provided slightly stronger staining and improved morphology compared to their air-dried counterparts.

For the remainder of the Examples the following conditions were used unless otherwise indicated.
1. Microscope slides containing tissues that were spray fixed.
2. Microscope slides containing tissues that had been stored at −20 C for not greater than 7 days.

TABLE 2
Results of Permeabilization of Skin Biopsies.

| Permeabilization Conditions | | | |
|---|---|---|---|
| Time | | Temperature | Staining Results for AE1/AE3 |
| Not Done | | Not Done | 2.0 |
| 1 | min | RT | 2.0 |
| 5 | min | RT | 2.0 |
| 30 | min | RT | 2.5 |
| 60 | min | RT | 3.0 |
| 1 | min | 90 C. | 3.0 |
| 5 | min | 90 C. | 3.0 |
| 30 | min | 90 C. | 3.0 |
| 60 | min | 90 C. | 3.0 |

Surprisingly we were able to achieve strong staining after only 1 minute incubation with the permeabilization reagent at 90 C. Having already achieved our goal of increasing staining, while only adding 1 minute to the overall protocol time, we did not test additional variables.

### Example 3

### Fc-Specific Secondary Antibody

The following secondary antibodies were tested:
1. Goat anti-mouse IgG (heavy and light chain specific) polymer with horseradish peroxidase (GAM-HRP polymer).
2. Goat anti-mouse IgG (Fc-specific) polymer with horseradish peroxidase (GAM-Fc-HRP polymer).

Slides with tissues were prepared and permeabilized according to Examples 1 and
2. Slides were stained according to Example 1 for AE1/AE3 except the GAM-Fc-HRP polymer was compared to the GAM-HRP polymer as follows:
1. AE1/AE3 primary antibody incubated for 20 minutes.
2. HRP polymer secondary antibody incubated for 20 minutes.
3. DAB chromogen incubated for 5 minutes.
4. Counterstained with Hematoxylin for 3 minutes.
5. Mounted and observed under the microscope.
6. Graded on a scale of 0 to 3.

TABLE 3
Results of Test of GAM-Fc-HRP polymer vs. GAM-HRP polymer
using sequential incubation.

| Secondary Antibody | Staining results for AE1/AE3 |
|---|---|
| GAM-HRP Polymer | 3.0 |
| GAM-Fc-HRP | 3.0 |
| Polymer | |

These results showed that both the GAM-HRP polymer and the GAM-Fc-HRP polymer performed comparably using a sequential incubation.

### Example 4

### Test of GAM-Fc-HRP Polymer vs. GAM-HRP Polymer Using Simultaneous Incubation

The following three conditions were tested:

1 Pre-mix the primary antibody and the secondary polymer, and then apply

to the tissue as a single step.

2. Apply the primary antibody onto the slide first and then apply the secondary polymer onto the tissue so that both reagents incubate simultaneously.
3. Apply the primary antibody onto the slide first, wait for a period of time and drain off excess primary antibody without rinsing, and then add the secondary polymer onto the tissue.

TABLE 4
Results of Test of various incubation methods for
GAM-Fc-HRP polymer.

| Secondary | Test | Staining results |
|---|---|---|
| Antibody | Conditions | for AE1/AE3 |
| GAM-HRP | Pre-mix with primary antibody | 0.0 |
| GAM-HRP | Apply primary antibody first | 0.5 |
| | Then apply secondary antibody | |
| GAM-HRP | Apply primary antibody first | 1.0 |
| | Drain Slide | |
| | Apply secondary antibody | |
| GAM-Fc-HRP | Pre-mix with primary antibody | 2.0 |
| GAM-Fc-HRP | Apply primary antibody first | 2.5 |
| | Then apply secondary antibody | |
| GAM-Fc-HRP | Apply primary antibody first | 3.0 |
| | Drain Slide | |
| | Apply secondary antibody | |

These results show that the GAM-Fc-HRP polymer reagent gave significantly stronger staining compared to the GAM-HRP polymer when the polymer reagent was allowed to react in the presence of the primary antibody.

### Example 5

### Rapid Dehydration and Mounting

A dehydrating reagent was prepared by preparing a solution of 10% polyvinyl in ethanol. After immunohistochemical staining and Hematoxylin counterstaining the slides were prepared for dehydration and mounting as follows:

### Standard Method of Dehydration.

1. Bath of 50% water and 50% alcohol for 3 min.
2. Bath of 25% water and 75% alcohol for 3 min.
3. Bath of 0% water and 100% alcohol for 3 min.
4. Bath of 50% alcohol and 50% xylene for 3 min.
5. Bath of 25% alcohol and 75% xylene for 3 min.
6. Bath of 0% alcohol and 100% xylene for 3 min.
7. One drop of resin-mounting medium in xylene.
8. Glass coverslip.

### Rapid Dehydration Method (Method 400).

1. Bath of dehydration reagent (dehydrating reagent prepared by preparing a solution of 10% polyvinyl in ethanol), quick dunk, 1-2 seconds.
2. Remove slides, drain, and air-dry, 10 seconds.
3. One drop of resin-mounting medium in xylene.
4. Glass coverslip.

TABLE 5
Results of Comparison of Rapid dehydration to standard dehydration.

| Method | Staining | Optical Resolution | Morphology |
|---|---|---|---|
| Standard | 3.0 | Excellent | Excellent. |
| Rapid | 3.0 | Excellent. | Excellent |

These results show that the rapid dehydration method (method 400) was equivalent to the standard dehydration method in terms of staining, optical resolution, and morphology. However, the rapid dehydration method took less than 20 seconds to complete compared to about 20 minutes for the standard dehydration method.

### Example 6

### Rapid Immunohistochemistry

Having completed all of the preliminary steps to develop a rapid immunohistochemistry staining method 100, the following protocol was tested to see whether the individual incubation times could be reduced with a goal of achieving a complete immunohistochemical stain within 10 minutes.
1. Permeabilization reagent was pre-heated in a microwave for 1 minute or until a temperature of approximately 90 C was reached. This step was performed while the slides were simultaneously being prepared, thus adding no additional time to the protocol.
2. Slides were placed into the pre-heated permeabilization reagent for 1 minute. This step was performed on a bench top at room temperature, while the permeabilization reagent cooled slightly.
3. Slides were transferred briefly to a buffer bath.
4. Slides were arranged horizontally and 100 ul of primary antibody was overlaid the tissue and incubated for 2.5 minutes.
5. Excess antibody was drained from the slide, and without rinsing 100 ul of GAM-Fc-HRP Polymer or GAR-Fc-HRP Polymer for mouse or rabbit primary antibodies respectively was overlaid the tissue and incubated for 2.5 minutes.
6. Slides were rinsed to remove excess primary antibody and polymer, then the tissues were overlaid with 100 ul DAB and incubated for 2.5 minutes.
7. Slides were rinsed in water and then counterstained with Hematoxylin for 10 seconds.
8. Slides were rinsed in water and then dehydrated with rapid dehydration reagent for 20 seconds.
9. Slides were mounted with mounting medium and coverslipped.
10. Total time of incubations was 9 minutes. Additional time for rinsing slides added approximately 1 additional minute for a total run time of about 10 minutes.

Table 6 shows a comparison of staining results comparing Rapid Immunohistochemistry of Example 6 to Standard Immunohistochemistry of Example 1.

TABLE 6
Comparison of Rapid Immunohistochemistry
to Standard Immunohistochemistry.

| | | | Staining Results | |
|---|---|---|---|---|
| | | | Immunohistochemistry | |
| Primary | Primary | Secondary | Method | |
| Antibody | Species | Antibody | Rapid | Standard |
| AE1/AE3 | Mouse | GAM-Fc-HRP Polymer | 3.0 | 3.0 |
| Ki67 | Rabbit | GAR-Fc-HRP Polymer | 3.0 | 3.0 |
| Vimentin | Rabbit | GAR-Fc-HRP Polymer | 3.0 | 3.0 |
| Melanoma | Mouse | GAM-Fc-HRP Polymer | 3.0 | 3.0 |
| (HMB45) | | | | |

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

what is claimed is:

1. A method for immunohistochemical detection of an antigen from a biological sample, the method comprising the steps of:
depositing a section of the biological sample on a slide;
permeabilizing the section of the biological sample by contacting the biological sample with a permeabilization reagent that comprises a Tris buffer, ethylenediaminetetraacetic acid, and a detergent;
incubating the section of the biological sample with a primary antibody to the antigen from the biological sample to bind the primary antibody to the antigen, the primary antibody having an Fc portion, and the primary antibody being one of a mouse antibody and a rabbit antibody;
incubating the section of the biological sample with a secondary antibody by simultaneously applying the primary antibody and the secondary antibody to the section of the biological sample to bind the secondary antibody with the Fc portion of the primary antibody, wherein the secondary antibody has a detectable label, and wherein the secondary antibody is one of;
i. anti-mouse Fc specific when the primary antibody is the mouse antibody; and
ii. anti-rabbit Fc specific when the primary antibody is the rabbit antibody;
removing unbound secondary antibody from the section of the biological sample;
mounting the section of the biological sample to the slide; and
detecting the secondary antibody bound to the antigen section of the biological sample.

2. The method of claim 1, wherein the section of the biological sample is produced by freezing the biological sample and then sectioning the biological sample.

3. The method of claim 1, wherein the section of the biological sample is produced by embedding the biological sample in paraffin and then sectioning the biological sample.

4. The method of claim 1, wherein the detectable label comprises an enzyme molecule.

5. The method of claim 1, wherein the detectable label comprises a polymer having two or more enzyme molecules bound to it.

6. The method of claim 1, wherein the detectable label comprises a fluorescent tag.

7. The method of claim 1, wherein the step of mounting the section of the biological sample to the slide comprises applying a mixture of an alcohol and an alcohol and water soluble polymer to the section of the biological sample.

8. The method of claim 1, wherein the secondary antibody is an antibody polymer.

9. The method of claim 1, wherein the primary antibody and the secondary antibody are premixed together before being simultaneously applied to the biological sample.

10. The method of claim 9, wherein the primary antibody and the secondary antibody are premixed together in a ratio of between 1:2.5 to 1:3.5 weight by weight of the primary antibody to Fc-specific secondary antibody.

11. A method for immunohistochemical detection of an antigen from a biological sample during a cancer removal procedure, the method comprising the steps of:
depositing a section of the biological sample on a slide, wherein the section of the biological sample is produced by freezing the biological sample and then sectioning the biological sample;
incubating the section of the biological sample with a primary antibody to the antigen from the biological sample to bind the primary antibody to the antigen, the primary antibody having an Fc portion, and the primary antibody being one of mouse antibody and rabbit antibody;
incubating the section of the biological sample with a secondary antibody by simultaneously applying the primary antibody and the secondary antibody to the section of the biological sample to bind the secondary antibody with the Fc portion of the primary antibody, wherein the primary antibody and the secondary antibody are premixed together in a ratio of between 1:2.5 to 1:3.5 weight by weight of the primary antibody to Fc-specific secondary antibody, wherein the secondary antibody has a detectable label, and wherein the secondary antibody is one of;
i. anti-mouse Fc specific when the primary antibody is the mouse antibody; and
ii. anti-rabbit Fc specific when the primary antibody is the rabbit antibody;
removing unbound secondary antibody from the section of the biological sample;
mounting the section of the biological sample to the slide, wherein the step of mounting the section of the biological sample to the slide comprises applying a mixture of an alcohol and an alcohol and water soluble polymer to the section of the biological sample; and
detecting the secondary antibody bound to the antigen in the section of the biological sample.

12. The method of claim 10, wherein the detectable label comprises an enzyme molecule.

13. The method of claim 10, wherein the detectable label comprises a polymer having two or more enzyme molecules bound to it.

14. The method of claim 10, wherein the detectable label comprises a fluorescent tag.

15. The method of claim 10, wherein the primary antibody and the secondary antibody are premixed together before being simultaneously applied to the biological sample.

16. The method of claim 10, wherein the secondary antibody is an antibody polymer.

17. The method of claim 10, further comprising the step of permeabilizing the section of the biological sample by contacting the biological sample with a permeabilization reagent that comprises a Tris buffer, ethylenediaminetetraacetic acid, and a detergent, wherein the step of permeabilizing the section of the biological sample by contacting the biological sample with a permeabilization reagent that comprises a Tris buffer, ethylenediaminetetraacetic acid, and a detergent is performed before incubating the section of the biological sample with the primary antibody.
